(19) United States
(12) Patent Application Publication
(10) Pub. No.: US 2004/0063670 A1
(43) Pub. Date: Apr. 1, 2004

(54) Use of bisphosphonates for pain treatment

(76) Inventors: Alyson Fox, London (GB); Jonathan Green, Arlesheim (CH); Terence O'Reilly, Basel (CH); Laszlo Urban, London (GB); Katharine Walker, Princeton, NJ (US)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/432,847

(22) PCT Filed: Nov. 27, 2001
(86) PCT No.: PCT/EP01/13836

(30) Foreign Application Priority Data
Nov. 29, 2000 (GB) 0029111.2

(51) Int. Cl. A61K 31/66

(52) U.S. Cl. 514/102

(57) ABSTRACT

A method for the treatment of pain, in particular antinociceptive or anti-allodynic treatment of pain, in a patient in need of such treatment, e.g. a patient with osteoporosis or osteopenia, a tumour patient or a patient suffering from an inflammatory disease, which comprises administering an effective amount of a bisphosphonate, e.g. zoledronic acid or salts or hydrates thereof, to the patient.

This invention relates to pharmaceutical compositions and uses, in particular to pharmaceutical compositions comprising bisphosphonates and to new therapeutic uses of bisphosphonates.

Bisphosphonates are widely used to inhibit osteoclast activity in a variety of both benign and malignant diseases which involve excessive or inappropriate bone resorption. These pyrophosphate analogs not only reduce the occurrence of skeletal related events but they also provide patients with clinical benefit and improve survival. Bisphosphonates are able to prevent bone resorption in vivo; the therapeutic efficacy of bisphosphonates has been demonstrated in the treatment of osteoporosis, osteopenia, Paget's disease of bone, tumour-induced hypercalcemia (TIH) and, more recently, bone metastases (BM) and multiple myeloma (MM) (for review see Fleisch H 1997 Bisphosphonates clinical. In Bisphosphonates in Bone Disease. From the Laboratory to the Patient. Eds: The Parthenon Publishing Group, New York/London pp 68-163). The mechanisms by which bisphosphonates inhibit bone resorption are still not completely understood and seem to vary according to the bisphosphonates studied. Bisphosphonates have been shown to bind strongly to the hydroxyapatite crystals of bone, to reduce bone turn-over and resorption, to decrease the levels of hydroxyproline or alkaline phosphatase in the blood, and in addition to inhibit the formation, recruitment, activation and the activity, of osteoclasts. Recently farnesyl diphosphate synthase, an enzyme of the mevalonate pathway of cholesterol biosynthesis, has been identified as the molecular target of nitrogen-containing bisphosphonates (reviewed in Rogers M J, Gordon S, Benford H L, Coxon F P, Luckman S P, Monkkonen J, Frith J C. 2000. Cellular and molecular mechanisms of action of bisphosphonates. Cancer 88(suppl):2961-2978)

Bone pain resulting from structural damage, periosteal irritation, and nerve entrapment is the most common complication of both benign and metastatic bone disease, and presents a significant problem in both hospital and community practice (Coleman, 1997, Cancer 80; 1588-1594).

MM is a plasma-cell malignancy characterized by the proliferation and the accumulation of malignant plasma cells within the bone marrow. The main clinical consequences are lytic bone lesions associated with pathologic fractures and bone pain. These lesions result from an excessive bone resorption, frequently leading to hypercalcemia. Bisphosphonates have been introduced for the long-term treatment of MM in combination with conventional chemotherapy. It has been shown recently that bisphosphonates such as clodronate and pamidronate can reduce the occurrence of skeletal related events such as lytic bone lesions and pathologic fractures and can relieve associated bone pain and improve the quality of life of patients (Laktinen et al. Lancet 1992, 340, 1049-1052; McCloskey et al. B. J. Haematol., 1998, 100, 317-325; and Berenson et al. N. Eng. J. Med. 1996, Vol. 334, No. 8, 488-493). Similar effects have been reported in breast cancer patients treated with bisphosphonates (Hortobagyi G N, Theriault R L, Porter L, Blayney D, Lipton A, Sinoff C, Wheeler H, Simeone J F, Seaman J, Knight R D. Efficacy of pamidronate in reducing skeletal complications in patients with breast cancer and lytic bone metastases. Protocol 19 Aredia Breast Cancer Study Group., N Engl J Med. 1996;335:1785-91; Kanis J A, Powles T, Paterson A H G, McCloskey E V, Ashley S. Clodronate decreases the frequency of skeletal metastases in women with breast cancer. Bone 1996; 19: 663-7.)

It has now been found surprisingly that certain bisphosphonates exert profound and apparently direct palliative effects on pain in in vivo animal models. For example, zoledronic acid has been found to reverse mechanical hyperalgesia in rat models of chronic inflammatory and neuropathic pain, with a fast onset of action and efficacy of up to about 100%. Additionally zoledronic acid has been found to reduce mechanical allodynia and reduce hind limb sparing in a rat model of bone cancer pain. These results indicate that zoledronic acid and similar bisphosphonates may have direct, fast acting, anti-nociceptive and anti-allodynic activity on pain.

Accordingly the present invention provides a method for the treatment of pain in a patient in need of such treatment, which comprises administering an effective amount of a bisphosphonate to the patient.

The invention further provides use of a bisphosphonate in the preparation of a medicament for the treatment of pain.

The invention yet further provides use of a bisphosphonate to treat pain associated with diseases or pathological conditions in mammals.

The present invention is particularly applicable to the palliative treatment of pain, i.e. the direct relief of pain in addition to the relief of pain as the result of amelioration of the underlying disease or medical condition, which is the cause of the pain. Thus, advantageously the invention provides methods and uses for the direct analgesic or acute treatment of pain.

Preferably the invention is used for the direct treatment of pain in diseases and medical conditions in which bisphosphonates are used to inhibit osteoclast activity. For example, the invention may be used for direct treatment of pain in diseases and conditions which involve excessive or inappropriate bone loss e.g. as the result of inappropriate osteoclast activity. Examples of such diseases and conditions include benign diseases and conditions such as osteoporosis of various genesis, Pagets disease, osteoarthritis, RA, periodontal disease; and especially malignant diseases such as MM and TIH and BM associated with various cancers, e.g. cancer of the breast, prostate, lung, kidney, ovary, or osteosarcoma. Generally the invention may be used to treat pain in other circumstances where bisphosphonates are used and pain is encountered, e.g. when bisphosphonates are use in bone fracture healing, osteonecrosis or treatment of prosthesis loosening.

The uses and methods of the present invention represent an improvement to existing therapy of malignant diseases in which bisphosphonates are used to prevent or inhibit development of bone metastases or excessive bone resorption, and also for the therapy of inflammatory diseases such as rheumatoid arthritis and osteoarthritis, as well as for all forms of osteoporosis and osteopenia.

Thus in the present description the terms “treatment” or “treat” refer to both prophylactic or preventative treatment as well as curative or palliative treatment of pain,in particular anti-nociceptive and anti-allodynic treatment of pain, especially treatment of bone pail

Thus in particular embodiments the invention provides:

a method for the treatment of bone pain in a patient in need of such treatment which comprises administering an effective amount of a bisphosphonate to the patient;

use of a bisphosphonate in the preparation of a medicament for the treatment of bone pain; or

use of a bisphosphonate as an agent for treatment of bone pain.

The bisphosphonates used in the present invention are typically those which relieve pain, in particular those which have an anti-nociceptive or anti-allodynic, and preferably rapid onset, activity on pain.

Thus, for example, suitable bisphosphonates for use in the invention may include the following compounds or a pharmaceutically acceptable salt thereof, or any hydrate thereof: 3-amino-1-hydroxypropane-1,1-diphosphonic acid (pamidronic acid), e.g. pamidronate (APD); 3-(N,N-dimethylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-ethidene-bisphosphonic acid, e.g. etidronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-n-pentylamino)-1-hydroxypropane-1,1-diphosphonic acid, e.g. methyl-pentyl-APD (=BM 21.0955); 1-hydroxy-2-(imidazol-1-yl)ethane-1,1-diphosphonic acid, e.g. zoledronic acid; 1-hydroxy-2-(3-pyridyl)ethane-1,1-diphosphonic acid (risedronic acid), e.g. risedronate, including N-methyl pyridinium salts thereof, for example N-methyl pyridinium iodides such as NE-10244 or NE-10446; 1-(4-chlorophenylthio)methane-1,1-diphosphonic acid (tiludronic acid), e.g. tiludronate; 3-[N-(2-phenylthioethyl)-N-methylamino]-1-hydroxypropane--1,1-diphosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-diphosphonic acid, e.g. FR 78844 (Fujisawa); 5-benzoyl-3,4dihydro-2H-pyrazole-3,3-diphosphonic acid tetraethyl ester, e.g. U-81581 (Upjohn); 1-hydroxy-2-(imidazo[1,2-a]pyridin-3-yl)ethane-1,1-diphosphonic acid, e.g. YM 529; and 1,1-dichloromethane-1,1-diphosphonic acid (clodronic acid), e.g. clodronate.

Preferably the bisphosphonates for use in the invention are the nitrogen containing bisphosphonates. For the purposes of the present descxription a nitrogen containing bisphosphonate is a compound which in addition to the characteristic geminal bisphosphate (P—C—P) moiety comprises a nitrogen containing side chain, e.g. a compound of formula I

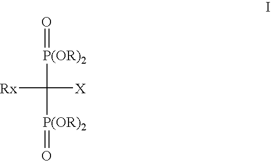

wherein

X is hydrogen, hydroxyl, amino, alkanoyl,or an amino group substituted by C₁-C₄ alkyl or alkanoyl;

R is hydrogen or C₁-C₄ alkyl and

Rx is a side chain which contains an optionally substituted amino group, or a nitrogen containing heterocycle (including aromatic nitrogen-containing heterocycles),

and pharmaceutically acceptable salts thereof or any hydrate thereof.

Particularly preferred nitorogen containing bisphosphonates are those having side chains containing nitrogen-containing heterocycles, most especially containing aromatic nitrogen-containing heterocycles.

Thus in one embodiment a particularly preferred bisphosphonate for use in the invention comprises a compound of Formula I′

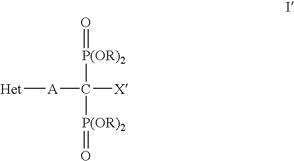

wherein

Het is an imidazole, oxazole, isoxazole, oxadiazole, thiazole, thiadiazole, pyridine, 1,2,3-triazole, 1,2,4-triazole or benzimidazole radical, which is optionally substituted by alkyl, alkoxy, halogen, hydroxyl carboxyl an amino group optionally substituted by alkyl or alkanoyl radicals or a benzyl radical optionally substituted by alkyl, nitro, amino or aminoalkyl;

A is a straight-chained or branched, saturated or unsaturated hydrocarbon moiety containing from 1 to 8 carbon atoms;

X is a hydrogen atom, optionally substituted by alkanoyl, or an amino group optionally substituted by alkyl or alkanoyl radicals, and

R is a hydrogen atom or a C₁-C₄ alkyl radical,

and the pharmacologically acceptable salts thereof.

In a further embodiment a particularly preferred bisphosphonate for use in the invention comprises a compound of Formula II

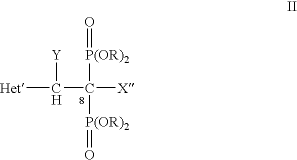

wherein

Het′ is a substituted or unsubstituted heteroaromatic five-membered ring selected from the group consisting of imidazolyl, imidazolinyl, isoxazolyl, oxazolyl, oxazolinyl, thiazolyl, thiazolinyl, triazolyl, oxadiazolyl and thiadiazolyl wherein said ring can be partly hydrogenated and wherein said substituents are selected from at least one of the group consisting of C₁-C₄ alkyl, C₁-C₄ alkoxy, phenyl, cyclohexyl, cyclohexylmethyl halogen and amino and wherein two adjacent alkyl substituents of Het can together form a second ring;

Y is hydrogen or C₁-C₄ alkyl;

X″ is hydrogen, hydroxyl amino, or an amino group substituted by C₁-C₄alkyl, and

R is hydrogen or C₁-C₄ alkyl;

as well as the pharmacologically acceptable salts and isomers thereof.

In a yet further embodiment a particularly preferred bisphosphonate for use in the invention comprises a compound of Formula III

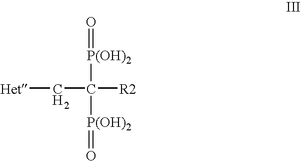

wherein

Het″ is an imidazolyl, 2H-1,2,3-, 1H-1,2,4- or 4H-1,2,4-triazolyl, tetrazolyl, oxazolyl, isoxazolyl, oxadiazolyl, thiazolyl or thiadiazolyl radical which is unsubstituted or C-mono- or di-substituted by lower alkyl, by lower alkoxy, bx phenyl which may in turn be mnon- or disubstituted by lower alkyl, lower alkoxy and/or halogen, by hydroxy, by di-lower alkylamino, by lower alkylthio and/or by halogen and is N-substituted at a substitutable N-atom by lower alkyl or by phenyl-lower alkyl which may in turn be mono- or di-substituted in the phenyl moiety by lower alkyl, lower alkoxy and/or halogen, and

R₂ is hydrogen, hydroxy, amino, lower alkylthio or halogen, lower radicals having up to and including 7 C-atoms,

or a pharmacologically acceptable salt thereof.

Examples of particularly preferred bisphophonates for use in the invention are:

2-(1-Methylimidazol-2-yl)-1-hydroxyethane-1,1-diphosphonic acid;

2-(1-Benzylimidazol-2-yl)-1-hydroxyethane-1,1-diphosphonic acid;

2-(1-Methylimidazol-4-yl)-1-hydroxyethane-1,1-diphosphonic acid;

1-Amino-2-(1-methylimidazol-4-yl)ethane-1,1-diphosphonic acid;

1-Amino-2-(1-benzylimidazol-4-yl)ethane-1,1-diphosphonic acid;

2-(1-Methylimidazol-2-yl)ethane-1,1-diphosphonic acid;

2-(1-Benzylimidazol-2-yl)ethane-1,1-diphosphonic acid;

2-(Imidazol-1-yl)-1-hydroxyethane-1,1-diphosphonic acid;

2-(Imidazol-1-yl)ethane-1,1-diphosphonic acid;

2-(4H-1 ,2,4-triazol-4-yl)-1-hydroxyethane-1,1-diphosphonic acid;

2-(Thiazol-2-yl)ethane-1,1-diphosphonic acid;

2-(Imidazol-2-yl)ethane-1,1-diphosphonic acid;

2-(2-Methylimidazol-4(5)-yl)ethane-1,1-diphosphonic acid;

2-(2-Phenylimidazol-4(5)-yl)ethane-1,1-diphosphonic acid;

2-(4,5-Dimethylimidazol-1-yl)-1-hydroxyethane-1,1-diphosphonic acid, and

2-(2-Methylimidazol-4(5)-yl)-1-hydroxyethane-1,1-diphosphonic acid,

and pharmacologically acceptable salts thereof.

The most preferred bisphosphonate for use in the invention is 2-(imidazol-1yl)-1-hydroxyethane-1,1-diphosphonic acid (zoledronic acid) or a pharmacologically acceptable salt thereof or any hydrate thereof.

Pharmacologically acceptable salts are preferably salts with bases, conveniently metal salts derived from groups Ia, Ib, IIa and IIb of the Periodic Table of the Elements, including alkali metal salts, e.g. potassium and especially sodium salts, or alkaline earth metal salts, preferably calcium or magnesium salts, and also ammonium salts with ammonia or organic amines.

Especially preferred pharmaceutically acceptable salts are those where one, two, three or four, in particular one or two, of the acidic hydrogens of the bisphosphonic acid are replaced by a pharmaceutically acceptable cation, in particular sodium, potassium or ammonium, in first instance sodium.

A very preferred group of pharmaceutically acceptable salts is characterized by having one acidic hydrogen and one pharmaceutically acceptable cation, especially sodium, in each of the phosphonic acid groups.

All the bisphosphonic acid derivatives mentioned above are well known from the literature. This includes their manufacture (see e.g. EP-A-513760, pp. 13-48). For example, 3-amino-1-hydroxypropane-1,1-diphosphonic acid is prepared as described e.g. in U.S. Pat. No. 3,962,432 as well as the disodium salt as in U.S. Pat. Nos. 4,639,338 and 4,711,880, and 1-hydroxy-2-(imidazol-1-yl)ethane-1,1-diphosphonic acid is prepared as described e.g. in U.S. Pat. No. 4,939,130. See also U.S. Pat. Nos. 4,777,163 and 4,687,767.

The bisphosphonates (hereinafter referred to as the Agents of the Invention) may be used in the form of an isomer or of a mixture of isomers where appropriate, typically as optical isomers such as enantiomers or diastereoisomers or geometric isomers, typically cis-trans isomers. The optical isomers are obtained in the form of the pure antipodes and/or as racemates.

The Agents of the Invention can also be used in the form of their hydrates or include other solvents used for their crystallisation.

The Agents of the Invention (the bisphosphonates) are preferably used in the form of pharmaceutical compositions that contain a therapeutically effective amount of active ingredient optionally together with or in admixture with inorganic or organic, solid or liquid, pharmaceutically acceptable carriers which are suitable for administration.

The pharmaceutical compositions may be, for example, compositions for enteral, such as oral, rectal, aerosol inhalation or nasal administration, compositions f6r parenteral, such as intravenous or subcutaneous administration, or compositions for transdermal administration (e.g. passive or iontophoretic).

Preferably, the pharmaceutical compositions are adapted to oral or parenteral (especially intravenous, intra-arterial or transdermal) administration. Intravenous and oral, first and foremost intravenous, administration is considered to be of particular importance. Preferably the bisphosphonate active ingredient is in the form of a parenteral, most preferably an intravenous form.

The particular mode of administration and the dosage may be selected by the attending physician taking into account the particulars of the patient, especially age, weight, life style, activity level, hormonal status (e.g. post-menopausal) and bone mineral density as appropriate. Most preferably, however, the bisphosphonate is administered intravenously.

The dosage of the Agents of the Invention may depend on various factors, such as effectiveness and duration of action of the active ingredient, mode of administration, warm-blooded species, and/or sex, age, weight and individual condition of the warm-blooded animal.

Normally the dosage is such that a single dose of the bisphosphonate active ingredient from 0.002-20.0 mg/kg, especially 0.01-10.0 mg/kg, is administered to a warm-blooded animal weighing approximately 75 kg. If desired, this dose may also be taken in several, optionally equal, partial doses.

“mg/kg” means mg drug per kg body weight of the mammal—including man—to be treated.

The dose mentioned above—either administered as a single dose (which is preferred) or in several partial doses—may be repeated, for example once daily, once weekly, once every month, once every three months, once every six months or once a year. In other words, the pharmaceutical compositions may be administered in regimens ranging from continuous daily therapy to intermittent cyclical therapy.

Preferably, the bisphosphonates are administered in doses which are in the same order of magnitude as those used in the treatment of the diseases classically treated with bisphosphonic acid derivatives, such as Paget's disease, tumour-induced hypercalcemia or osteoporosis. In other words, preferably the bisphosphonic acid derivatives are administered in doses which would likewise be therapeutically effective in the treatment of Paget's disease, tumour-induced hypercalcaemia or osteoporosis, i.e. preferably they are administered in doses which would likewise effectively inhibit bone resorption. For example, for the preferred nitrogen-containing bisphosphonates, e.g. zoledronic acid and salts thereof, doses of bisphosphonate in the range from about 0.5 to about 20 mg, preferably from about 1 to about 10 mg, may be used for treatment of human patients.

Formulations in single dose unit form contain preferably from about 1% to about 90%, and formulations not in single dose unit form contain preferably from about 0.1% to about 20%, of the active ingredient. Single dose unit forms such as capsules, tablets or dragees contain e.g. from about 1 mg to about 500 mg of the active ingredient.

Pharmaceutical preparations for enteral and parenteral administration are, for example, those in dosage unit forms, such as dragees, tablets or capsules and also ampoules. They are prepared in a manner known per se, for example by means of conventional mixing, granulating, confectioning, dissolving or lyophlising processes.

For example, pharmaceutical-preparations for oral administration can be obtained by combining the active ingredient with solid carriers, where appropriate granulating a resulting mixture, and processing the mixture or granulate, if desired or necessary after the addition of suitable adjuncts, into tablets or dragée cores. Suitable carriers are especially fillers, such as sugars, for example lactose, saccharose, mannitol or sorbitol, cellulose preparations and/or calcium phosphates, for example tricalcium phosphate or calcium hydrogen phosphate, and also binders, such as starch pastes, using, for example, corn, wheat, rice or potato starch, gelatin, tragacanth, methylcellulose and/or polyvinylpyrrolidone and, if desired, disintegrators, such as the above-mentioned starches, also carboxymethyl starch, crosslinked polyvinylpyrrolidone, agar or alginic acid or a salt thereof, such as sodium alginate. Adjuncts are especially flow-regulating agents and lubricants, for example silicic acid, talc, stearic acid or salts thereof, such as magnesium or calcium stearate, and/or polyethylene glycol. Dragee cores are provided with suitable coatings that may be resistant to gastric juices, there being used, inter alia, concentrated sugar solutions that optionally contain gum arabic, talc, polyvinylpyrrolidone, polyethylene glycol and/or titanium dioxide, or lacquer solutions in suitable organic solvents or solvent mixtures or, to produce coatings that are resistant to gastric juices, solutions of suitable cellulose preparations, such as acetylcellulose phthalate or hydroxypropylmethylcellulose phthalate. Colouring substances or pigments may be added to the tablets or dragee coatings, for example for the purpose of identification or to indicate different doses of active ingredient.

Other orally administrable pharmaceutical preparations are dry-filled capsules made of gelatin, and also soft, sealed capsules made of gelatin and a plasticiser, such as glycerol or sorbitol. The dry-filled capsules may contain the active ingredient in the form of a granulate, for example in admixture with fillers, such as lactose, binders, such as starches, and/or glidants, such as talc or magnesium stearate, and, where appropriate, stabilisers. In soft capsules the active ingredient is preferably dissolved or suspended in suitable liquids, such as fatty oils, paraffin oil or liquid polyethylene glycols, it being possible also for stabilisers to be added.

Parenteral formulations are especially injectable fluids that are effective in various manners, such as intra-arterially, intramuscularly, intraperitoneally, intranasally, intradermally, subcutaneously or preferably intravenously. Such fluids are preferably isotonic aqueous solutions or suspensions which can be prepared before use, for example from lyophilised preparations which contain the active ingredient alone or together with a pharmaceutically acceptable carrier. The pharmaceutical preparations may be sterilised and/or contain adjuncts, for example preservatives, stabilisers, wetting agents and/or emulsifiers, solubilisers, salts for regulating the osmotic pressure and/or buffers.

Suitable formulations for transdermal application include an effective amount of the active ingredient with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the active ingredient of the skin of the host at a controlled and predetermined rate over a prolonged period of time, and means to secure the device to the skin.

The following Examples illustrate the invention described hereinbefore.

In the following Examples the term “active ingredient” is to be understood as being any one of the bisphosphonic acid derivatives mentioned above as being useful according to the present invention.

### EXAMPLES

### Example 1

Capsules containing coated pellets of active ingredient, for example, disodium pamidronate pentahydrate, as active ingredient:

| | |
|---|---|
| Core pellet: | |
| active ingredient (ground) | 197.3 mg |
| Microcrystalline cellulose | 52.7 mg |
| (Avicel ® PH 105) | |
| | 250.0 mg |
| + Inner coating: | |
| Cellulose HP-M 603 | 10.0 mg |
| Polyethylene glycol | 2.0 mg |
| Talc | 8.0 mg |
| | 270.0 mg |
| + Gastric juice-resistant outer coating: | |
| Eudragit ® L 30 D (solid) | 90.0 mg |
| Triethyl citrate | 21.0 mg |
| Antifoam ® AF | 2.0 mg |
| Water | |
| Talc | 7.0 mg |
| | 390.0 mg |

A mixture of disodium pamidronate with Avicel® PH 105 is moistened with water and kneaded, extruded and formed into spheres. The dried pellets are then successively coated in the fluidized bed with an inner coating, consisting of cellulose HP-M 603, polyethylene glycol (PEG) 8000 and talc, and the aqueous gastric juice-resistant coat, consisting of Eudragit® L 30 D, triethyl citrate and Antifoam AF. The coated pellets are powdered with talc and filled into capsules (capsule size 0) by means of a commercial capsule filling machine, for example Höfliger and Karg.

### Example 2

Monolith adhesive transdermal system, containing as active ingredient, for example, 1-hydroxy-2-(imidazol-1-yl)-ethane-1,1-diphosphonic acid:

| Composition: | |
|---|---|
| polyisobutylene (PIB) 300 | 5.0 g |
| (Oppanol B1, BASF) | |
| PIB 35000 | 3.0 g |
| (Oppanol B10, BASF) | |
| PIB 1200000 | 9.0 g |
| (Oppanol B100, BASF) | |
| hydrogenated hydrocarbon resin | 43.0 g |
| (Escorez 5320, Exxon) | |
| 1-dodecylazacycloheptan-2-one | 20.0 g |
| (Azone, Nelson Res., Irvine/CA) | |
| active ingredient | 20.0 g |
| Total | 100.0 g |

The above components are together dissolved in 150 g of special boiling point petroleum fraction 100-125 by rolling on a roller gear bed. The solution is applied to a polyester film (Hostaphan, Kalle) by means of a spreading device using a 300 mm doctor blade, giving a coating of about 75 g/m². After drying (15 minutes at 60° C.), a silicone-treated polyester film (thickness 75 mm, Laufenberg) is applied as the peel-off film. The finished systems are punched out in sizes in the wanted form of from 5 to 30 cm² using a punching tool. The complete systems are sealed individually in sachets of aluminised paper.

### Example 3

Vial containing 1.0 mg dry, lyophilized 1-hydroxy-2-(imidazol-1-yl)ethane-1,1-diphosphonic acid (mixed sodium salts thereof). After dilution with 1 ml of water, a solution (concentration 1 mg/ml) for i.v. infusion is obtained.

| Composition: | | | |
|---|---|---|---|
| active ingredient (free diphosphonic acid) | | 1.0 | mg |
| mannitol | | 46.0 | mg |
| Trisodium citrate × 2 H₂O | ca. | 3.0 | mg |
| water | | 1 | ml |
| water for injection | | 1 | ml. |

In 1 ml of water, the active ingredient is titrated with trisodium citrate×2 H₂O to pH 6.0. Then, the mannitol is added and the solution is lyophilized and the lyophilisate filled into a vial.

### Example 4

Ampoule containing active ingredient, for instance disodium pamidronate pentahydrate dissolved in water. The solution (concentration 3 mg/ml) is for i.v. infusion after dilution.

| Composition: | | |
|---|---|---|
| active ingredient | 19.73 | mg |
| ( [symbol] 5.0 mg of anhydrous active ingredient) | | |
| mannitol | 250 | mg |
| water for injection | 5 | ml. |

### Example 5

### The Effect of Bisphosphonates in Rat Models of Inflammatory and Neuropathic Pain

Methods

Inflammatory hyperalgesia

Mechanical hyperalgesia was examined in a rat model of inflammatory pain. Paw withdrawal thresholds to an increasing pressure stimulus were measured by the Randal-Sellito technique using an analgesymeter (Ugo Basile, Milan), in naïve animals prior to an intraplantar injection of complete Freund's complete adjuvant (FCA) into the left hind paw. 24 h later paw withdrawal thresholds were measured again prior to (predose) and then from 10 min to 6 h following drug or vehicle administration. Reversal of hyperalgesia in the ipsilateral paw was calculated according to the formula:

$$\% reversal = \frac{postdose threshold - predose threshold}{naive threshold - predose threshold} \times 100$$

Neuropathic hyperalgesia

Mechanical hyperalgesia was examined in a rat model of neuropathic pain induced by partial ligation of the left sciatic nerve. Approximately 14 days following surgery mechanical withdrawal thresholds of both the ligated (ipsilateral) and non-ligated (contralateral) paw were measured prior to (predose) and then from 10 min to 6 h following drug or vehicle administration. Reversal of hyperalgesia at each time point was calculated according to the formula:

$$\% reversal = \frac{ipsilateral threshold postdose - ipsilateral threshold predose}{contralateral threshold predose - ipsilateral threshold predose} \times 100$$

All experiments were carried out using groups of 6 animals. Stock concentrations of drugs were dissolved in distilled water and subsequent dilutions were made in 0.9% saline for subcutaneous administration in a volume of 4 mlkg⁻¹. All drugs were made up in plastic vials and kept in the dark.

Statistical analysis was carried out on withdrawal threshold readings (g) using ANOVA with repeated measures followed by Tukey's HSD test. Efficacy refers to the maximal reversal of hyperalgesia observed at the doses used.

Results

1. In a model of inflammatory hyperalgesia induced by unilateral hindpaw injection of complete Freund's adjuvant Zoledronate (0.003-0.1 mgkg⁻¹ s.c.) produced a dose-dependant reversal of mechanical hyperalgesia. The effect was rapid in onset, with a maximal reversal of 100% within 30 min, and of short duration with no significant activity 3 h following administration. Some contralateral activity was observed at the highest dose.

2. Pamidronate (0.03-1mgkg⁻¹ s.c.) and Clodronate (0.3-10 mgkg⁻¹ s.c.) were both ineffective in reversing inflammatory mechanical hyperalgesia, but rather produced slight reductions of paw withdrawal thresholds at the highest doses tested.

3. In a model of chronic neuropathic pain induced by unilateral partial sciatic nerve ligation. Zoledronate (0.003-0.1 mgkg⁻¹ s.c.) produced a moderate 40% reversal of mechanical hyperalgesia which was maximal within 30 min of administration. However, there was also a significant reduction in contralateral paw withdrawal thresholds at the highest dose.

4. Pamidronate (0.03-1 mgkg⁻¹ s.c.) was only weakly active in the model of neuropathic pain, producing a maximal 20% reversal of hyperalgesia, whilst Clodronate (0.3-10 mgkg⁻¹ s.c.) was inactive. Both drugs again produced some reductions in contraleral paw withdrawal thresholds.

5. These data show that Zoledronate reverses mechanical hyperalgesia in models of chronic inflammatory and neuropathic pain in the rat.

### Example 6

The Effect of Bisphosphonates in a Rat Model of Bone Cancer Pain

Adult female rats were given intra-tibial injections of MRMZ1 rat mammary gland carcinoma cells (3 μl, 10⁷ cells/ml). These animals gradually developed mechanical hyperalgesia, mechanical allodynia (skin sensitivity to non-noxious stimuli) and hind limb sparing, beginning on day 12-14 following cell injection. Zoledronic acid (ZOL) (10 and 30 μg/kg s.c.) administered 3 times a week from the day of cell injection, produced a profound inhibition of hind limb sparing and mechanical allodynia In comparison to vehicle-treated controls, which showed maximal hind limb sparing by day 19, rats given the higher ZOL dose did not develop any sign of hind limb sparing over 19 days following intra-tibial cell injection. However, when administered as a single injection (100 μg/kg, s.c.) on day 19, ZOL had no acute effect. By contrast, acute treatment with morphine (1-10 mg/kg, s.c.) produced a dose dependent reduction in mechanical allodynia and, at the highest dose only, also a significant reduction in hind limb sparing.

1. A method for the treatment of pain in a patient in need of such treatment which comprises administering an effective amount of a bisphosphonate to the patient.

2. Use of a bisphosphonate in the preparation of a medicament for the treatment of pain.

3. Use of a bisphosphonate to treat pain associated with diseases or pathological conditions in mammals.

4. A method for the anti-nociceptive or anti-allodynic treatment of pain in a patient in need of such treatment which comprises administering an effective amount of a bisphosphonate to the patient;
use of a bisphosphonate in the preparation of a medicament for the anti-nociceptive or anti-allodynic treatment of pain; or
use of a bisphosphonate as an anti-nociceptive or anti-allodynic agent.

5. A method for the treatment of bone pain in a patient in need of such treatment which comprises administering an effective amount of a bisphosphonate to the patient;
use of a bisphosphonate in the preparation of a medicament for the treatment of bone pain; or
use of a bisphosphonate as an agent for treatment of bone pain.

6. A method according to claim 1 or a use according to claim 2 or 3 for the treatment of pain associated with osteoporosis, rheumatoid arthritis, osteoarthritis and tumour formation, e.g. tumour growth, invasion or metastasis.

7. A method according to claim 1 or a use according to claim 2 or 3, in which the bisphosphonate is selected from the following compounds or a pharmaceutically acceptable salt thereof, or any hydrate thereof: 3-amino-1-hydroxypropane-1,1-diphosphonic acid (pamidronic acid), e.g. pamidronate (APD); 3-(N,N-dimethylamino)1-hydroxypropane--1,1-diphosphonic acid, e.g. dimethyl-APD; 4-amino-1-hydroxybutane-1,1-diphosphonic acid (alendronic acid), e.g. alendronate; 1-hydroxy-ethidene-bisphosphonic acid, e.g. etidronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-n-pentylamino)-1-hydroxypropane-1,1-diphosphonic acid, e.g. methyl-pentyl-APD (=BM 21.0955); 1-hydroxy-2-(imidazol-1-yl)ethane-1,1-diphosphonic acid; 1-hydroxy-2-(3-pyridyl)ethane-1,1-diphosphonic acid (risedronic acid), e.g. risedronate, including N-methyl pyridinium salts thereof, for example N-methyl pyridinium iodides such as NE-10244 or NE-10446; 1-(4chlorophenylthio)methane-1,1-diphosphonic acid (tiludronic acid), e.g. tiludronate; 3-[N-(2-phenylthioethyl)-N-methylamino]-1-hydroxy-propane-1,1-diphosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-diphosphonic acid, e.g. PR 78844 (Fujisawa); 5-benzoyl-3,4dihydro-2H-pyrazole-3,3-diphosphonic acid tetraethyl ester, e.g. U-81581 (Upjohn); 1-hydroxy-2-(imidazo[1,2-a]pyridin-3-yl)ethane--1,1-diphosphonic acid, e.g. YM 529; and 1,1-dichloromethane-1,1-diphosphonic acid (clodronic acid), e.g. clodronate.

8. A method according to claim 1 or a use according to claim 2 or 3, in which the bisphosphonate is a compound of Formula III 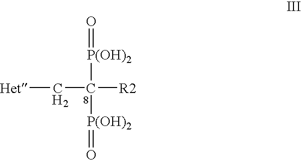
wherein
Het″ is an imidazolyl, 2H-1,2,3-, 1H-1,2,4- or 4H-1,2,4-triazolyl, tetrazolyl, oxazolyl, isoxazolyl, oxadiazolyl, thiazolyl or thiadiazolyl radical which is unsubstituted or C-mono- or di-substituted by lower alkyl, by lower alkoxy, bx phenyl which may in turn be mnon- or disubstituted by lower alkyl, lower alkoxy and/or halogen, by hydroxy, by di-lower alkylamino, by lower alkylthio and/or by halogen and is N-substituted at a substitutable N-atom by lower alkyl or by phenyl-lower alkyl which may in turn be mono- or di-substituted in the phenyl moiety by lower alkyl, lower alkoxy and/or halogen, and
R₂ is hydrogen, hydroxy, amino, lower alkylthio or halogen, lower radicals having up to and including 7 C-atoms,
or a pharmacologically acceptable salt thereof.

9. A method according to claim 1 or a use according to claim 2 or 3, in which the bisphosphonate is zoledronic acid, or a pharmaceutically acceptable salt thereof, or any hydrate thereof.

10. All novel compounds, processes, methods and uses substantially as hereinbefore described with particular reference to the Examples.
